(19) United States
(12) Patent Application Publication
(10) Pub. No.: US 2026/0183446 A1
(43) Pub. Date: Jul. 2, 2026

(54) PROTECTIVE ADHESIVE COMPOSITION FOR DIGESTIVE TRACT MUCOSA AND APPLICATION THEREOF

(71) Applicant: Bi-trumed Biotech Co., Ltd., Haining City (CN)

(72) Inventors: Fei GAO, Haining City (CN); Weike LU, Haining City (CN); Yanli WU, Haining City (CN); Yi HONG, Haining City (CN); Xinyi GAO, Haining City (CN)

(73) Assignee: Bi-trumed Biotech Co., Ltd., Haining City (CN)

(21) Appl. No.: 19/004,088

(22) Filed: Dec. 27, 2024

(51) Int. Cl. A61L 24/04; A61L 24/02; A61L 24/08; A61L 24/10

(52) U.S. Cl. CPC A61L 24/043; A61L 24/02; A61L 24/08; A61L 24/10; A61L 2430/22

(57) ABSTRACT

Provided is a protective adhesive composition for digestive tract mucosa and application thereof, belonging to the technical field of biomedicine. The protective adhesive composition includes an adhesive-forming substance, a cross-linking agent, an adhesive agent, and an acid-base regulator. The protective adhesive composition may form a colloidal membrane, and has acid-resisting and pepsin-resisting effects, such that a stimulating effect of digestive fluids on injured mucosa is avoided, thereby promoting the healing of wounds.

### FIELD

The present disclosure belongs to the technical field of biomedicine, and in particular, relates to a protective adhesive composition for digestive tract mucosa and an application thereof.

### BACKGROUND

Digestive tract mucosa includes esophageal mucosa, intestinal mucosa, and gastric mucosa. The gastric mucosa is a thin layer of mucosal tissue in the stomach of a human body, consists of epithelium, lamina propria, and muscularis mucosae, and continuously secrets three key substances of gastric acid, pepsinogen, and gastric mucus, so as to cause the stomach to play an important role in digestion, absorption, sterilization, self-protection, etc.

Acid-related diseases (e.g., gastritis, peptic ulcer, etc.) are caused by imbalance between mucosal invasion factors (including gastric acid, pepsin, abnormal motility, cholic acid, certain drugs, and Hp) and mucosal protective factors (including mucosal barriers, mucosal blood flow, bicarbonate secretion, and epithelial regeneration). Therefore, the treatment of the acid-related diseases is mainly to inhibit the digestion of acids and protease to the gastrointestinal mucosa, that is, to protect the gastrointestinal mucosa, so as to avoid the self-digestion of the mucosa, thereby promoting the healing of ulcer.

In addition to protective adhesives for drugs, in order to promote the rapid healing of wounds in recent years, various wound protective adhesives have been developed, which may be classified into traditional protective adhesives, biological protective adhesives, artificially-synthesized protective adhesives, growth factor protective adhesives, etc. However, existing wound protective adhesives tend to adhere to the wound, causing mechanical damage to the wound when being replaced, or are poor in strength and easy to fall off from the wound, or are susceptible to erosion by digestive fluids. Therefore, a wound protective adhesive that can form a gel protective layer and is strong in elasticity, excellent in viscosity, and resistant to acid erosion is an urgent problem that needs to be solved by those skilled in the art.

### SUMMARY

In the present disclosure, in view of disadvantages in the related art, a first objective of the present disclosure is to provide a protective adhesive composition for digestive tract mucosa. The protective adhesive composition consists of an adhesive-forming substance, a cross-linking agent, an adhesive agent, and an acid-base regulator, the adhesive-forming substance is pectin and alginate, the cross-linking agent is one of aluminum bicarbonate, aluminum phosphate, or calcium phosphate, the adhesive agent is chitosan or deacetylated chitosan and polypeptide; the acid-base regulator is one of carbonate, bicarbonate, phosphate, or citrate.

Preferably, the protective adhesive composition is consisting of the adhesive-forming substance, the cross-linking agent, the adhesive agent, and the acid-base regulator; the adhesive-forming substance is the pectin and the alginate; the cross-linking agent is the aluminum bicarbonate; the adhesive agent is the chitosan and the polypeptide; the acid-base regulator is the bicarbonate.

Preferably, a weight ratio of the adhesive-forming substance:the cross-linking agent:the adhesive agent is 1:0.1-0.3:0.2-0.5; and the amount of the acid-base regulator used is to regulate pH at 7.0-7.5.

Preferably, the pectin is a low ester pectin.

Preferably, a degree of esterification of the low ester pectin is 5-20%.

Preferably, the alginate is sodium alginate, potassium alginate, or ammonium alginate.

Preferably, a mass ratio of the pectin:the alginate is 0.5-1:1-2.

Preferably, a weight ratio of the chitosan or deacetylated chitosan:the polypeptide is 1:0.5-1.

Preferably, the polypeptide is sodium polyglutamate, polylysine, or polyaspartate.

Preferably, the polylysine is F-polylysine; and preferably, a molecular weight of the F-polylysine is 1000-6000 daltons.

Preferably, the acid-base regulator is sodium bicarbonate, sodium carbonate, potassium carbonate, dipotassium hydrogen phosphate, disodium hydrogen phosphate, trisodium phosphate, tripotassium phosphate, sodium citrate, potassium citrate, or monosodium citrate.

Another objective of the present disclosure is to provide an application of the protective adhesive composition for digestive tract mucosa.

The composition may be used in the form of solutions, gels, gums, emulsions, or ointments.

The application of the protective adhesive composition for digestive tract mucosa is preferably an application in preparation of drugs for treating and protecting peptic ulcer in gastric mucosa.

The protective adhesive composition for digestive tract mucosa may be preferably used on gastric mucosa through an endoscope; and the composition forms a gel physical protection membrane by being used on the gastric mucosa.

Another objective of the present disclosure is to provide an application method of the protective adhesive composition for digestive tract mucosa.

A method for treating peptic ulcer includes applying an effective dose of the protective adhesive composition for digestive tract mucosa to a patient required to be treated, so as to form a protective layer on digestive tract mucosal tissue.

Compared with the related art, the present disclosure has the following beneficial effects.

Compared to conventional adhesive agents such as cellulose-sodium carboxymethyl cellulose/carboxymethyl cellulose and the like, the adhesive agent in the present disclosure is the chitosan or deacetylated chitosan and the polypeptide, which has stronger adhesion. In particular, the polylysine binds tightly to target tissue and a slime layer through the interaction of positive ion groups on its side chain, and thus has stronger adhesion.

Furthermore, under the synergistic effect of the adhesive-forming substance and the cross-linking agent, better effects of resisting gastric acid and pepsin are achieved; and the healing of digestive tract mucosa is promoted by accelerating the proliferation of mucosal cells and migration towards wounds.

### DESCRIPTION OF EMBODIMENTS

The present disclosure is described in further detail below with reference to the drawings and specific implementations, and the following materials are commercially available.

### Example 1

Low ester pectin (degree of esterification being 6%), 0.6 g
Sodium alginate, 1.4 g
Aluminum bicarbonate, 0.5 g
Chitosan, 0.2 g
Sodium polyglutamate, 0.2 g

The above components were dissolved by using 100 ml distilled water, pH was regulated to 7.2 with a sodium bicarbonate solution, each bottle was packed with 6 ml, and high-temperature steam sterilization was performed.

### Example 2

Low ester pectin (degree of esterification being 6%), 1 g
Sodium alginate, 1.8 g
Aluminum bicarbonate, 0.3 g
Chitosan, 0.8 g
ε-polylysine, 0.6 g

The above components were dissolved by using 100 ml distilled water, pH was regulated to 7.2 with a sodium bicarbonate solution, each bottle was packed with 6 ml, and high-temperature steam sterilization was performed.

### Example 3

Low ester pectin (degree of esterification being 6%), 2 g
Sodium alginate, 4 g
Aluminum bicarbonate, 1 g
Deacetylated chitosan, 1.8 g
ε-polylysine, 1 g

The above components were dissolved by using 100 ml distilled water, pH was regulated to 7.2 with a sodium bicarbonate solution, each bottle was packed with 6 ml, and high-temperature steam sterilization was performed.

### Example 4

Low ester pectin (degree of esterification being 6%), 2.5 g
Sodium alginate, 4.5 g
Aluminum bicarbonate, 1.4 g
Deacetylated chitosan, 2 g
Polyaspartate, 1 g

The above components were dissolved by using 100 ml distilled water, pH was regulated to 7.2 with a sodium bicarbonate solution, each bottle was packed with 6 ml, and high-temperature steam sterilization was performed.

### Example 5

Low ester pectin (degree of esterification being 6%), 3 g
Sodium alginate, 4 g
Aluminum bicarbonate, 1 g
Deacetylated chitosan, 1 g
Sodium polyglutamate, 0.7 g

The above components were dissolved by using 100 ml distilled water, pH was regulated to 7.2 with a sodium bicarbonate solution, each bottle was packed with 6 ml, and high-temperature steam sterilization was performed.

### Example 6

Low ester pectin (degree of esterification being 12%), 4 g
Sodium alginate, 6 g
Aluminum phosphate, 3 g
Chitosan, 1 g
Polyaspartate, 1 g

The above components were dissolved by using 100 ml distilled water, pH was regulated to 7.2 with a sodium bicarbonate solution, each bottle was packed with 6 ml, and high-temperature steam sterilization was performed.

### Example 7

Low ester pectin (degree of esterification being 12%), 2.5 g
Sodium alginate, 2.5 g
Calcium phosphate, 1.5 g
Chitosan, 1.5 g
ε-polylysine, 1 g

The above components were dissolved by using 100 ml distilled water, pH was regulated to 7.2 with a sodium bicarbonate solution, each bottle was packed with 6 ml, and high-temperature steam sterilization was performed.

### Comparative Example 1

A difference between this comparative example and Example 1 lied in that, low ester pectin (degree of esterification being 24%), 0.6 g, with the rest being the same.

### Comparative Example 2

A difference between this comparative example and Example 2 lied in that: high ester pectin (degree of esterification being 55%), 1 g, with the rest being the same.

### Comparative Example 3

Low ester pectin (degree of esterification being 12%), 2.5 g
Sodium alginate, 2.5 g
Aluminum phosphate, 1 g
Carboxymethyl cellulose, 1.5 g
Polyaspartate, 0.8 g

The above components were dissolved by using 100 ml distilled water, pH was regulated to 7.2 with a sodium bicarbonate solution, each bottle was packed with 6 ml, and high-temperature steam sterilization was performed.

### Comparative Example 4

Low ester pectin (degree of esterification being 12%), 6 g
Sodium alginate, 6 g
Calcium sulfate, 3.5 g
Carboxymethyl cellulose, 2 g
ε-polylysine, 2 g

The above components were dissolved by using 100 ml distilled water, pH was regulated to 7.2 with a sodium bicarbonate solution, each bottle was packed with 6 ml, and high-temperature steam sterilization was performed.

### Comparative Example 5

Low ester pectin (degree of esterification being 6%), 4 g
Sodium alginate, 5 g
Sodium bicarbonate, 2 g
Carboxymethyl cellulose, 1.2 g
ε-polylysine, 0.9 g

The above components were dissolved by using 100 ml distilled water, pH was regulated to 7.2 with a sodium bicarbonate solution, each bottle was packed with 6 ml, and high-temperature steam sterilization was performed.

### Comparative Example 6

Low ester pectin (degree of esterification being 6%), 2 g
Sodium alginate, 8 g
Sodium bicarbonate, 2 g
Bletilla striata polysaccharide, 3 g
ε-polylysine, 2 g

The above components were dissolved by using 100 ml distilled water, pH was regulated to 7.2 with a sodium bicarbonate solution, each bottle was packed with 6 ml, and high-temperature steam sterilization was performed.

### Biocompatibility Research Test

1. Oral mucosal irritation: normal saline was added according to a proportion of 0.2 g/ml, so as to prepare a test solution. In particular, test solutions of samples in the above Examples and comparative examples were prepared into cotton balls with a diameter of about 6 mm, and the cotton balls were soaked and placed into one-side cheek pouches of 3 golden hamsters. Contact time was at least 5 min each time, and the above irritation was repeated every 24 h, with a total of 5 times; the cheek pouches were observed with naked eyes after 24 h from the last contact; the hamsters were killed without pain; tissue samples from representative sites of the cheek pouches were taken and placed into a 6% formaldehyde solution, fixed, and prepared into tissue sections, and then histological evaluation was performed to calculate stimulation indexes.

Results: the stimulation indexes in the above Examples and comparative examples were all 0, and no oral mucosal irritation was observed in the tested samples.

2. Cytotoxicity: a culture medium preparation test solution was added according to a proportion of 0.2 g/ml. Then, determination was performed according to a cytotoxicity test specified in Appendix C of GB/T16886.5-2017 Biological evaluation of medical devices Part 5: In vitro cytotoxicity test, that is, an MMT method.

Results: the cytotoxicity in the above Examples and comparative examples were all within a range of Level 0-1.

3. Sensitization test: the normal saline was added according to a proportion of 0.2 g/ml, so as to prepare a test solution. Then, a skin sensitization test was performed according to a method specified in GB/T16886.10-2017 Biological Evaluation of Medical Devices Part 10: Irritation and Skin Sensitization Tests.

Results: no sensitization was observed in the above Examples and comparative examples.

### Performance Test for Protective Adhesive Composition

### 1. Acid Sensitivity Test

(1) 6 ml of colloid solutions prepared in the above Examples and comparative examples were poured into aseptic culture dishes with diameters being 6 cm, then 10 ml of artificial gastric juices (hydrochloric acid solutions without pepsin) with pH=1.0 were added and cured for 30 minutes, 60 minutes, 120 minutes, and 180 minutes, and excess solutions were poured off. Samples were prepared into 2 cm×2 cm pieces, 3 pieces were taken at each time point so as to determine tensile strength, and an average value of the tensile strength at each time point in the above Examples and comparative examples was calculated, referring to Table 1.

TABLE 1
Tensile strength (N) at different curing
times for pH = 1.0 hydrochloric acid

| | 30 | 60 | 120 | 180 |
|---|---|---|---|---|
| | minutes | minutes | minutes | minutes |
| Example 1 | 0.122 | 0.183 | 0.263 | 0.383 |
| Example 2 | 0.203 | 0.240 | 0.279 | 0.438 |
| Example 3 | 0.148 | 0.161 | 0.211 | 0.359 |
| Example 4 | 0.132 | 0.183 | 0.242 | 0.360 |
| Example 5 | 0.182 | 0.228 | 0.268 | 0.347 |
| Example 6 | 0.165 | 0.193 | 0.253 | 0.373 |
| Example 7 | 0.180 | 0.216 | 0.257 | 0.347 |
| Comparative example 1 | 0.073 | 0.083 | 0.159 | 0.184 |
| Comparative example 2 | 0.084 | 0.095 | 0.17 | 0.196 |
| Comparative example 3 | 0.062 | 0.071 | 0.148 | 0.173 |
| Comparative example 4 | 0.070 | 0.082 | 0.157 | 0.17 |
| Comparative example 5 | 0.071 | 0.080 | 0.181 | 0.175 |
| Comparative example 6 | 0.071 | 0.081 | 0.179 | 0.172 |

Test results showed that, when pH=1.0, if the curing time was longer, the tensile strength was stronger, a tensile force was uneven when the curing time was 30 min, the adhesive broke easily; as the curing time prolonged, the tensile strength increased, and the colloid solution was uneasy to break; and under the same conditions, the tensile strength in the Examples were stronger than that in the comparative examples.

(2) 6 ml of the colloid solutions prepared in the above Examples and comparative examples were poured into the aseptic culture dishes with diameters being 6 cm, then 10 ml of artificial gastric juices (hydrochloric acid solutions without pepsin) with pH=1.0, 2.0, 3.0, and 4.0 were added and cured for 60 minutes, and excess solutions were poured off. The samples were prepared into 2 cm×2 cm pieces, 3 pieces were taken at each pH point so as to determine the tensile strength, and an average value of the tensile strength of hydrochloric acid curing at different pH values at the same time in the above Examples and comparative examples was calculated, referring to Table 2.

TABLE 2
Tensile strength (N) of hydrochloric acid
curing at different pH values at same time

| | 1.0 | 2.0 | 3.0 | 4.0 |
|---|---|---|---|---|
| Example 1 | 0.122 | 0.119 | 0.11 | 0.09 |
| Example 2 | 0.203 | 0.202 | 0.19 | 0.179 |
| Example 3 | 0.148 | 0.142 | 0.138 | 0.138 |
| Example 4 | 0.132 | 0.129 | 0.124 | 0.117 |
| Example 5 | 0.182 | 0.102 | 0.096 | 0.096 |
| Example 6 | 0.165 | 0.157 | 0.147 | 0.117 |
| Example 7 | 0.18 | 0.16 | 0.16 | 0.101 |
| Comparative example 1 | 0.073 | 0.061 | 0.055 | 0.052 |
| Comparative example 2 | 0.084 | 0.063 | 0.054 | 0.053 |
| Comparative example 3 | 0.062 | 0.053 | 0.049 | 0.047 |
| Comparative example 4 | 0.07 | 0.06 | 0.04 | 0.04 |
| Comparative example 5 | 0.071 | 0.069 | 0.059 | 0.045 |
| Comparative example 6 | 0.071 | 0.064 | 0.055 | 0.045 |

Test results showed that, after 60 min of curing, the impact of changes in the pH values was found to be that, colloid was easier to break when the pH value was greater than 2.0, and as a result, the tensile strength gradually reduced; and under the same conditions, the strength of the formed colloid in the Examples was superior to that in the comparative examples.

By combining the two results, it might be seen that, for the protective adhesive composition in the Examples, as the pH reduced and the time prolonged, the strength of the formed colloid was larger; and the material was acid-sensitive and had the better performance compared to the comparative examples.

### 2. Anti-Enzyme Degradation Test

6 ml of the colloid solutions prepared in Examples 1-7 and Comparative examples 1 and 2 were poured into the aseptic culture dishes with diameters being 6 cm, then 10 ml of artificial gastric juices (hydrochloric acid solutions without pepsin) with pH=1.2 were added to react for 30 minutes, and excess solutions were poured off. The samples were prepared into wafers with diameters being 16 mm (3 wafers were taken and dried to original weight, and then an average value was taken for calculation), every 3 wafers were soaked in the artificial gastric juice with an enzyme and the artificial gastric juice without an enzyme, the juices were changed every two days, and treatment was performed for 15 days at 37° C. The percentage of remaining mass the material at Day 3, Day 9, and Day 15 was determined, respectively, SPSS21.0 software was used, independent sample T-test were used for statistical analysis, and average degradation rates without and with pepsin at different days in the above Examples and comparative examples were calculated referring to Table 3.

The artificial gastric juice with an enzyme was a sodium chloride hydrochloride solution with pepsin of which pH was about 1.2; and the artificial gastric juice without an enzyme was a sodium chloride hydrochloride solution without pepsin of which pH was about 1.2.

TABLE 3
Degradation rates without and with pepsin at different days

| | Original value (g) | | Day 3 (%) | | Day 9 (%) | | Day 15 (%) | |
|---|---|---|---|---|---|---|---|---|
| | Without | With | Without | With | Without | With | Without | With |
| Example 1 | 0.0215 | 0.0211 | 44.28 | 44.04 | 28.89 | 29.42 | 18.55 | 18.54 |
| Example 2 | 0.0257 | 0.0263 | 44.11 | 44.20 | 34.46 | 34.71 | 18.72 | 18.55 |
| Example 3 | 0.0254 | 0.0254 | 43.74 | 43.79 | 30.33 | 30.52 | 20.07 | 19.98 |
| Example 4 | 0.0235 | 0.0235 | 44.09 | 44.18 | 34.44 | 34.69 | 18.70 | 18.53 |
| Example 5 | 0.0246 | 0.0247 | 44.27 | 44.03 | 28.88 | 29.41 | 18.54 | 18.53 |
| Example 6 | 0.0242 | 0.0244 | 43.94 | 43.90 | 34.47 | 34.72 | 18.73 | 18.67 |
| Example 7 | 0.0239 | 0.0238 | 44.24 | 44.00 | 28.97 | 29.41 | 18.25 | 18.24 |
| Comparative | 0.0229 | 0.0228 | 40.68 | 32.73 | 25.69 | 15.49 | 16.15 | 6.39 |
| example 1 | | | | | | | | |
| Comparative | 0.0225 | 0.0221 | 40.57 | 32.59 | 27.26 | 17.29 | 16.62 | 6.94 |
| example 2 | | | | | | | | |

Test results showed that, under the same conditions, the anti-enzyme degradation performance in Examples 1-7 was superior to that in Comparative examples 1 and 2. In Examples 1-7, through independent sample t testing, p>0.05, there was no significant difference, that is, there was no large difference between the degradation rates of a group without protease and a group with the protease, indicating that the samples were not degraded by the enzyme. Under the action of the pepsin, the wound protective adhesive for digestive tract mucosa was not degraded, and thus the protective adhesive had anti-enzyme degradation performance.

### 3. In Vitro Colloidal Membrane Formation Test

6 ml of the above solutions were respectively taken and coated on inner side surfaces of fresh pigskins, then the artificial gastric juice with dilute hydrochloric acid was sprayed, and the formation of the colloidal membranes was observed. Results were that colloidal membranes were formed in Examples 1-7 and Comparative examples 1-6.

### 4. Tissue Adhesion Test

Fresh pig stomach available on the market was taken and placed in the normal saline (37° C.), then the stomach was cut open, the lining of the stomach was cleaned with the normal saline, and the cleaned stomach was used within 2 hours. A certain area of stomach tissue (5 cm×5 cm) was cut and fixed on a polyethylene film, a 1 cmxl cm wound formed by a mucosal layer of the stomach was cut off, and 6 ml of the protective adhesive in Examples 1-7 and Comparative examples 1-6 was uniformly coated on the gastric mucosal wound and normal mucosa nearby. The stomach tissue was placed in a constant temperature and humidity chamber with a relative humidity of 90% and a temperature of 37° C. for 20 minutes, the treated stomach tissue was fixed on a washing inclined groove, an angle of the inclined groove was adjusted to 60 degrees, a flow rate of a peristaltic pump was adjusted to 20 ml/min, the stomach tissue was washed for 5 minutes with 0.1 mol/L hydrochloric acid, the wash solution was collected in an evaporating dish with known weight, and a certain area of the stomach tissue (5 cm×5 cm) was additionally taken, and the wash solution was collected through the same operations. Drying was performed at 70° C., weighing was performed, and tissue adhesion was represented by adhesion percentage. A calculation method was as follows:

$$Stomach tissue adhesion percentage ( Bg / \% ) Bg / \% = \{ [ M - ( G - g - m ) ] / M \} \times 100 \%$$

In the formula, M was the dry amount of the protective adhesive composition for digestive tract mucosa; g was the weight of an empty evaporating dish; G was the total weight of the evaporating dish and dried residues; and m was the amount (blank control) of solids in the same volume of the wash solution, in g. Test results were shown in Table 4 (adhesion rate, in %), and the adhesion rate of the protective adhesive was greater than 93.5%.

TABLE 4
Tissue adhesion test results

| | Examples | | | | | | | Comparative examples | | | | | |
|---|---|---|---|---|---|---|---|---|---|---|---|---|---|
| / | 1 | 2 | 3 | 4 | 5 | 6 | 7 | 1 | 2 | 3 | 4 | 5 | 6 |
| Adhesion | 93.7 | 94.0 | 94.0 | 93.5 | 93.8 | 93.8 | 93.7 | 80.5 | 80.2 | 76.1 | 73.2 | 75.0 | 75.2 |
| rate | | | | | | | | | | | | | |

### Effectiveness Test for Protective Adhesive Composition in Animals

### 1. Animal Test

SD rats were grouped, and each group had 10 rats. The rats were fasted 24 hours before surgery, but could drink water. All surgical instruments were placed in a steam sterilizer for 20 min at 121° C., and then placed in a 37° C. oven for drying. The rats were anesthetized by intramuscular injection with Zoletil, and placed on an operating table on their backs with abdominal hair removal. A test region was sterilized with 2% iodine and a 75% ethanol solution according to routine surgical requirements. The skin, muscle layer, and peritoneum were cut at upper abdomen layer by layer with a scalpel, and if there was bleeding, ligaturing was performed to stop the bleeding. The stomach was exposed and cut open at lesser curvature of stomach; the stomach was washed with normal saline; 0.2 ml of the normal saline was injected under the mucosa at greater curvature in the stomach, so as to form a gastric mucosal protrusion; the mucosa with similar areas was cut off by using a snare to form wounds; complete hemostasis was performed with an electric knife; no treatment was performed on a control group; the protective adhesives in the above Examples and comparative examples were coated in a test group, and then the stomach was sutured after photographing. Then abdominal walls were sutured layer by layer. The rat was put in a feeding cage, and fasted for one day. One animal on the day of the test in the control group was taken for pathology to observe the depth of the wound.

The rat was dissected 10 days after surgery, the wound was photographed, image data was inputted into a computer, and Image J software was used to calculate the area of the wound, so as to obtain a wound healing rate. Wound healing rate=(original wound area-unhealed wound area)/original wound area×100%. Test results were shown in Table 5.

TABLE 5
Wound healing status

| | Bleeding situation | Wound healing rate |
|---|---|---|
| | (number of rats) | (%) |
| Control group | 5 | 32 |
| Example 1 | None | 97 |
| Example 2 | None | 100 |
| Example 3 | None | 99 |
| Example 4 | None | 96 |
| Example 5 | None | 98 |
| Example 6 | None | 98 |
| Example 7 | None | 97 |
| Comparative example 1 | None | 82 |
| Comparative example 2 | None | 82 |
| Comparative example 3 | 3 | 80 |
| Comparative example 4 | 2 | 70 |
| Comparative example 5 | None | 72 |
| Comparative example 6 | 4 | 71 |

Test results showed that, 10 days of gastric mucosa dissection, the Example group, the comparative example group and the control group were observed, the animals were in good status, and hair-standing phenomena were shown in some animals in the control group, and were not shown in the Example group and the comparative example group. The rats were dissected to observe ulcer surfaces after being killed with CO₂, and it was found that the ulcer surfaces of five rats were in a bleeding state, which was none in the Example group, and the control groups 3, 4, and 6 had different bleeding situations. By counting the wound areas, it was found that healing rate values of the ulcer surfaces of the rats in the control group were about 32%, the healing rate of the protective adhesive in the Example group was more than 90%, which was higher than that in the control group, such that the overall healing rate of the protective adhesive groups in the Example group was better than that in control group.

### 2. Proliferation and Migration Effects of Protective Adhesive on Epithelial Cells of Gastric Mucosa

(1) Preparation of cell culture medium: DMEM culture solution (high sugar) culture medium, 10 g/mL of bovine insulin, 50 mg/mL of gentamicin, and 10% fetal bovine serum were supplemented. In the Examples and comparative examples, 6 mL of samples were added to the cell culture solution and immersed for 24 hours at 37° C.; and liquid portions were taken, filtered with a 0.22 m microporous filter membrane, then sterilized; and kept in a 4° C. fridge in a light avoidance manner for later use.

(2) Cell culture: human gastric mucosal epithelial cells GES-1 were placed in a 37° C. aseptic incubator with saturated humidity and 5% CO₂ for culture, the cell culture solution were changed every 2-3 d, and passaged every 3-4 d to maintain a cell concentration at a degree of fusion of 70%-80%.

(3) Impact on proliferation of the gastric mucosal epithelial cells: a modeling process of cell proliferation referred to methods of relevant literature. Details were as follows: logarithmic growth phase cells were taken, a concentration thereof was adjusted to 2.0×10⁴ cell/mL; the cells were inoculated in a 96-well plate, with 100 μL in each well, and placed in the 37° C. aseptic incubator with saturated humidity and 5% CO₂ to culture for 24 h, and then the culture solution was replaced; 200 μL of an extract in each of the above Examples and comparative examples was added in the test group, a blank control group was the cell culture solution, and a solvent blank group was a corresponding component culture solution; and 6 holes were parallelly set in each group, culture was performed for 24 h and 48 h, then 20 μL of an MTS solution was added, continuous culture was performed for 4 h, absorbance (A492) was measured, and a cell proliferation rate was calculated.

$$Cell proliferation rate = ( Atest - Asolvent blank ) / ( Acontorl group - Asolvent blank ) \times 100 \% .$$

TABLE 6
Cell proliferation rate

| | 24 h (%) | 48 h (%) |
|---|---|---|
| Blank control group | 100 | 100 |
| Example 1 | 209 | 232 |
| Example 2 | 225 | 236 |
| Example 3 | 220 | 228 |
| Example 4 | 206 | 219 |
| Example 5 | 215 | 232 |
| Example 6 | 212 | 230 |
| Example 7 | 205 | 218 |
| Comparative example 1 | 158 | 165 |
| Comparative example 2 | 159 | 168 |
| Comparative example 3 | 155 | 164 |
| Comparative example 4 | 154 | 162 |
| Comparative example 5 | 152 | 164 |
| Comparative example 6 | 153 | 165 |

Test results showed that, although the proliferation effect of gastric mucosal cells was promoted in the Examples and comparative examples, the proliferation rate in the Examples was much higher than that in the comparative examples, and was twice as high as that of the blank control group.

(4) Impact on migration of the gastric mucosal epithelial cells: a modeling process of cell migration referred to methods of relevant literature. Details were as follows: for the human gastric mucosal epithelial cells at a logarithmic growth phase, the cells were inoculated in a 24-well plate with 400 μL of 6×10⁵ cells each well, the plate was placed in a 37° C. incubator with 5% CO₂ to culture for 24 h; after the cells were tightly connected to form a monolayer, a vertical mark was scratched in the center of the culture dish with a 1.5 mm diameter capillary tube, and both sides of the wound might be seen at the same time under a microscope. The original culture solution was sucked after scratching, each well was washed twice with a 200 μL Phosphate Buffer Solution (PBS) so as to remove unattached cells; then 400 μL of the extract of each of the Examples and comparative examples was added to each well, and 400 μL of the DMEM culture solution was only added to the control group; software Image pro plus 6.0 was used to measure the healing situations of the scratched wounds at 0, 6, 12, 24 h.

$$Cell migration rate = ( wound area at 0 h - wound area at T h ) / ( wound area at 0 h ) \times 100 \% .$$

TABLE 7
Cell migration rate

| | 6 h (%) | 12 h (%) | 24 h (%) |
|---|---|---|---|
| Control group | 6 | 18 | 40 |
| Example 1 | 32 | 65 | 92 |
| Example 2 | 38 | 69 | 96 |
| Example 3 | 35 | 68 | 96 |
| Example 4 | 30 | 63 | 93 |
| Example 5 | 33 | 67 | 94 |
| Example 6 | 32 | 66 | 93 |
| Example 7 | 31 | 64 | 91 |
| Comparative example 1 | 15 | 30 | 72 |
| Comparative example 2 | 16 | 31 | 74 |
| Comparative example 3 | 16 | 38 | 71 |
| Comparative example 4 | 10 | 35 | 66 |
| Comparative example 5 | 13 | 36 | 69 |
| Comparative example 6 | 14 | 38 | 70 |

Test results showed that, the migration of the gastric mucosal epithelial cells could be promoted in the Examples, and the migration rate was higher than that in the comparative examples and the control group.

Based on the above in vitro and in vivo research results, it indicated that the Example group of the present disclosure was high in component safety and good in acid resistance and pepsin resistance, and could significantly promote the proliferation and migration of the gastric mucosal epithelial cells; by accelerating the proliferation of the mucosal cells and the migration to the wound, irritation of digestive fluid to the wound was avoided, local inflammatory response of the wound was relieved, and tissue regeneration was promoted, thereby promoting the healing of digestive tract mucosa.

The specific embodiments described herein are merely illustrative of the spirit of the present disclosure. Those skilled in the art to which the present disclosure belongs may make a variety of modifications or additions to the specific embodiments described or use similar ways to replace them, but do not deviate from the spirit of the present disclosure.

What is claimed is:

1. A protective adhesive composition for digestive tract mucosa, consisting of an adhesive-forming substance, a cross-linking agent, an adhesive agent, and an acid-base regulator, wherein
the adhesive-forming substance is pectin and alginate;
the cross-linking agent is one of aluminum bicarbonate, aluminum phosphate, or calcium phosphate;
the adhesive agent is chitosan or deacetylated chitosan and polypeptide; and
the acid-base regulator is one of carbonate, bicarbonate, phosphate, or citrate.

2. The composition according to claim 1, consisting of the adhesive-forming substance, the cross-linking agent, the adhesive agent, and the acid-base regulator, wherein
the adhesive-forming substance is pectin and alginate;
the cross-linking agent is the aluminum bicarbonate;
the adhesive agent is the chitosan and the polypeptide; and
the acid-base regulator is the bicarbonate.

3. The composition according to claim 1, a weight ratio of the adhesive-forming substance:the cross-linking agent:the adhesive agent is 1:0.1-0.3:0.2-0.5; and the amount of the acid-base regulator used is to regulate pH at 7.0-7.5.

4. The composition according to claim 1, wherein a mass ratio of the pectin:the alginate is 0.5-1:1-2.

5. The composition according to claim 1, wherein the pectin is a low ester pectin.

6. The composition according to claim 5, wherein a degree of esterification of the low ester pectin is 5-20%.

7. The composition according to claim 1, wherein the alginate is sodium alginate, potassium alginate, or ammonium alginate.

8. The composition according to claim 1, wherein a weight ratio of the chitosan or deacetylated chitosan:the polypeptide is 1:0.5-1.

9. The composition according to claim 1, wherein the polypeptide is sodium polyglutamate, polylysine, or polyaspartate.

10. The composition according to claim 9, wherein the polylysine is F-polylysine.

11. The composition according to claim 1, wherein the acid-base regulator is sodium bicarbonate, sodium carbonate, potassium carbonate, dipotassium hydrogen phosphate, disodium hydrogen phosphate, trisodium phosphate, tripotassium phosphate, sodium citrate, potassium citrate, or sodium dihydrogen citrate.

12. The composition according to claim 1, wherein the composition is in the form of solutions, gels, gums, emulsions, or ointments.

13. An application of the composition according to claim 1 in preparation of drugs for treating and protecting peptic ulcer in digestive tract mucosa.

14. The composition according to claim 1, being used on gastric mucosa through an endoscope, wherein the composition forms a gel physical protection membrane when being used on the gastric mucosa.

15. A method for treating peptic ulcer, comprising:
administering an effective dose of the protective adhesive composition for digestive tract mucosa according to claim 1 to a patient in need of treatment, so as to form a protective layer on digestive tract mucosal tissue.
